(19) United States
(12) Patent Application Publication
(10) Pub. No.: US 2005/0182071 A1
(43) Pub. Date: Aug. 18, 2005

(54) Methods and systems for inhibiting arrhythmia

(75) Inventors: Kirk Patrick Seward, Dublin, CA (US); Lynn Mateel Barr, Lafayette, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: EndoBionics, Inc., San Leandro, CA (US)

(21) Appl. No.: 10/930,711

(22) Filed: Aug. 30, 2004

Related U.S. Application Data
(60) Provisional application No. 60/503,560, filed on Sep. 16, 2003.

(51) Int. Cl. A61K 31/4965

(52) U.S. Cl. 514/255.06

(57) ABSTRACT

Methods and systems for treating patients suffering from or at risk of cardiac arrhythmias rely on the injection of amiodarone and other class III anti-arrhythmic drugs into the perivascular space surrounding a cardiac blood vessel. Injection may be achieved using intravascular catheters which advance needles radially outward from a blood vessel lumen or by transmyocardial injection from an epicardial surface of the heart.

### CROSS-REFERENCES TO RELATED APPLICATIONS

This is an application claiming the benefit under 35 USC 119(e) of U.S. Provisional Patent Application Ser. No. 60/503,560 (Attorney Docket No. 021621-001900), filed Sep. 16, 2003, the full disclosure of which is incorporated herein by reference.

### BACKGROUND OF THE INVENTION

1. Filed of the Invention

The present invention relates generally to medical methods and devices. More particularly, the present invention relates to methods and systems for treating and inhibiting cardiac arrhythmias by the direct injection of a class III anti-arrhythmic drug into cardiac tissue.

Abnormal heart rhythms are referred to generally as arrhythmias. Arrhythmias may be characterized by increased heart rates, referred to as tachycardias, or by slower heart rates, referred to as bradycardias. Arrhythmias may occur in the atria, ventricles, or both. Generally, ventricular tachycardias are the most dangerous to the patient, although atrial arrhythmias are also problematic.

A variety of intravascular and pharmaceutical therapies have been developed for treating cardiac arrhythmias. For example, cardiac ablation catheters have been developed for altering the conductive pathways on the endocardial surfaces within the heart chambers. Alternatively, a variety of sodium channel blockers, calcium channel blockers, and beta blockers are now available for drug-based inhibition of cardiac arrhythmias and related conditions. Although both the catheter-based and pharmaceutical approaches have been effective, each suffer form shortcomings, and alternative and improved treatment modalities remain desirable.

Of particular interest to the present invention, amiodarone has been an anti-arrhythmic drug in wide spread use since the 1970s. It is a class III anti-arrhythmic drug, and is widely used in the treatment of ventricular tachycardias. It also possesses class I, class II, and class IV actions which affords a unique pharmacological and anti-arrhythmic profile. While amiodarone has been found particularly suitable for treating patients after acute myocardial infarction and/or after cardiac surgery during the period where patients are at increased risk of having fatal arrhythmias, the drug has significant side effects that make systemic treatment difficult. Moreover, as the onset of effectiveness of the drug is generally slow, it can be difficult to achieve the desired pharmakinetic profiles.

For these reasons, it would be desirable to provide improved methods and systems for delivering amiodarone and other class III anti-arrhythmic agents to patients, particularly to patients who have recently suffered an acute myocardial infarction or have recently undergone cardiac surgery. It would be particularly desirable if such methods and systems delivered the amiodarone and/or other agents directly to cardiac tissue, preferably to most or all tissues which can benefit from such drug treatment. Such methods and systems will preferably be catheter-based and permit introduction of the amiodarone and other agents into cardiac and other tissue near the coronary and peripheral vasculature, including both arteries and veins, should further provide delivery of such agents to precisely controlled locations within or adjacent to the target tissues, and should still further provide for the direct delivery of such agents into tissue without dilution in the systemic circulation. Further preferably, the methods and system should allow for the injection of the amiodarone and other agents in the tissue surrounding the coronary and peripheral vasculature in regions which permit the rapid and wide spread migration and distribution of the agents to remote regions of cardiac tissue in amounts and at levels sufficient to provide the desired therapeutic benefits. At least some of these objectives will be met by the inventions described hereinafter.

### BRIEF SUMMARY OF THE INVENTION

The present invention provides improved methods and systems for treating patients at risk of or suffering from cardiac arrhythmias, including tachycardias, bradycardias, and other arrhythmias which occur in either or both of the ventricles and/or atria. Methods and systems will be particularly suitable for treating patients who have recently suffered from an acute myocardial infarction (AMI), who have undergone cardiac surgery, including open chest surgery, closed chest surgery, stopped heart surgery, beating heart surgery, and variations thereof. Methods and systems of the present invention rely on the direct delivery of anti-arrhythmic drugs and biological agents, including cells, usually class III anti-arrhythmic drugs, particularly amiodarone, to cardiac tissue, usually employing a catheter for injection of the drugs through the endothelium of a cardiac artery or vein into the perivascular space beyond the outside of the external elastic lamina so that the drug is able to permeate through the vessel wall and into the adventitia.

The preferred amiodarone drugs utilized in the methods of the present invention are described in detail in Sloskey (1883) Clin. Pharm. 2:330-40 and Doggrell (2001) Expert Opin Pharmacother. 2:1877-90. Other class III anti-arrhythmic drugs and still other anti-arrhythmics useful in the present invention are well described in the medical literature, e.g., in Nacarelli et al. (2003) Am. J. Cardiol. 91:150-260.

A particular advantage of the present invention is the ability to deliver the class III anti-arrhythmic drug widely throughout the cardiac tissue with only one or a limited number of injections. It is presently believed that such wide distribution of the drug is best achieved when the drug is delivered into the perivascular space at a depth (measured from the interior of the associated blood vessel) which is within an annular space or envelope having a width from 10% to 50% of the vessel diameter measured from the exterior of the vessel. Typically, the annular envelope around the blood vessel into which the drug is to be injected will have a width in the range from 0.1 mm to 5 mm, preferably from 0.2 mm to 3 mm, with the greater widths corresponding to larger vessel diameters.

It is further believed that the wide distribution of the drug throughout the cardiac tissue may result from entry of the drug into the lymphatic system which surrounds the individual blood vessels. While this understanding of the potential mechanism of action may help understand and define the present invention, the present invention in no way depends on the accuracy of understanding this mechanism of distribution.

The methods and systems of the present invention preferably utilize injection from an intravascular device in order to deliver the class III anti-arrhythmic drugs to the perivascular space as defined above. Use of intravascular delivery is particularly preferred with those patients who are not undergoing procedures which would result in either open chest, intercostal, thoracoscopic or other direct access to the epicardial surface. One such direct access is provided, however, the methods of the present invention may be performed by injection transmyocardially from an epicardial surface to the target perivascular space surrounding the blood vessel. Accurate positioning of the needle may be achieved using, for example, transesophogeal imaging, flouroscopic imaging, or the like.

In particular, the preferred intravascular injection methods of the present invention comprise injecting a class III anti-arrhythmic drug into the adventitial and perivascular tissues by advancing a needle from a lumen of a cardiac blood vessel to the target location beyond the endothelium. The class III anti-arrhythmic drug is then delivered through the needle to the target tissues. The needle is at least into the perivascular space beyond the outside of the endothelium of the blood vessel, and usually is advanced into the adventitia surrounding the blood vessel.

The class III anti-arrhythmic drugs will be injected under conditions and in an amount sufficient to permeate circumferentially around the perivascular space of the blood vessel and into the adventitia over an axial length of the blood vessel of at least about 1 cm, usually at least about 2 cm, and more usually at least 3 cm, 5 cm, 10 cm, or greater. Thus, the needle may be advanced in a radial direction to a depth in the tissue surrounding the blood vessel equal to at least 10% of the mean luminal diameter of the blood vessel at the site of direct injection, more typically being in the range from 10% to 150%, usually from 10% to 50% of the mean luminal diameter.

Systems according to the present invention for treating a patient suffering from a cardiac arrhythmia comprise an amount of a class III anti-arrhythmic drug, particularly an amiodarone, sufficient to treat the heart and an intravascular catheter having a needle for injecting the drug into a location beyond the endothelium of the blood vessel as described above.

### BRIEF DESCRIPTION OF THE DRAWINGS

FIG. 1A is a schematic, perspective view of an intravascular injection catheter suitable for use in the methods and systems of the present invention.

FIG. 1B is a cross-sectional view along line 1B-1B of FIG. 1A.

FIG. 1C is a cross-sectional view along line 1C-1C of FIG. 1A.

FIG. 2A is a schematic, perspective view of the catheter of FIGS. 1A-1C shown with the injection needle deployed.

FIG. 2B is a cross-sectional view along line 2B-2B of FIG. 2A.

FIG. 3 is a schematic, perspective view of the intravascular catheter of Figs. 1A-1C injecting drug into an adventitial space surrounding a coronary blood vessel in accordance with the methods of the present invention.

FIG. 4 is a schematic, perspective view of another embodiment of an intravascular injection catheter useful in the methods of the present invention.

FIG. 5 is a schematic, perspective view of still another embodiment of an intravascular injection catheter useful in the methods of the present invention, as inserted into a patient's vasculature.

FIGS. 6A and 6B are schematic views of other embodiments of an intravascular injection catheter useful in the methods of the present invention (in an unactuated condition) including multiple needles.

FIG. 7 is a schematic view of yet another embodiment of an intravascular injection catheter useful in the methods of the present invention (in an unactuated condition).

FIG. 8 is a perspective view of a needle injection catheter useful in the methods and systems of the present invention.

FIG. 9 is a cross-sectional view of the catheter FIG. 8 shown with the injection needle in a retracted configuration.

FIG. 10 is a cross-sectional view similar to FIG. 9, shown with the injection needle laterally advanced into luminal tissue for the delivery of drug according to the present invention.

### DETAILED DESCRIPTION OF THE INVENTION

The present invention provides methods and systems for treating patients at risk of or suffering from cardiac arrhythmias. In particular, these patients will have been diagnosed or otherwise determined to be suffering from a tachycardia, bradycardia, or other cardiac arrhythmia relating to aberrant electrical conduction within the heart. In other cases, however, patients who have recently suffered from an acute myocardial infarction (AMI) or who have or will be undergoing cardiac surgery may also be candidates for receiving treatment according to the present invention in order to reduce the risk associated with future cardiac arrhythmias.

The present invention will preferably utilize microfabricated devices and methods for intravascular injection of the drug. The following description provides several representative embodiments of microfabricated needles (microneedles) and macroneedles suitable for the delivery of the drug into a perivascular space or adventitial tissue. The perivascular space is the potential space between the outer surface and the endothelium or “vascular wall” of either an artery or vein. The microneedle is usually inserted substantially normal to the wall of a vessel (artery or vein) to eliminate as much trauma to the patient as possible. Until the microneedle is at the site of an injection, it is positioned out of the way so that it does not scrape against arterial or venous walls with its tip. Specifically, the microneedle remains enclosed in the walls of an actuator or sheath attached to a catheter so that it will not injure the patient during intervention or the physician during handling. When the injection site is reached, movement of the actuator along the vessel terminated, and the actuator is operated to cause the microneedle to be thrust outwardly, substantially perpendicular to the central axis of a vessel, for instance, in which the catheter has been inserted.

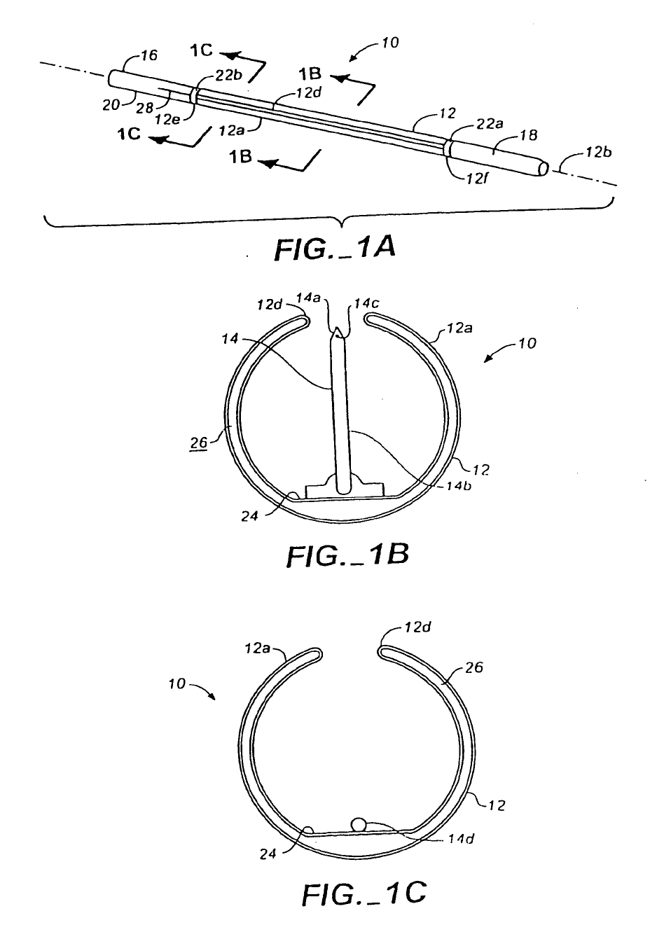

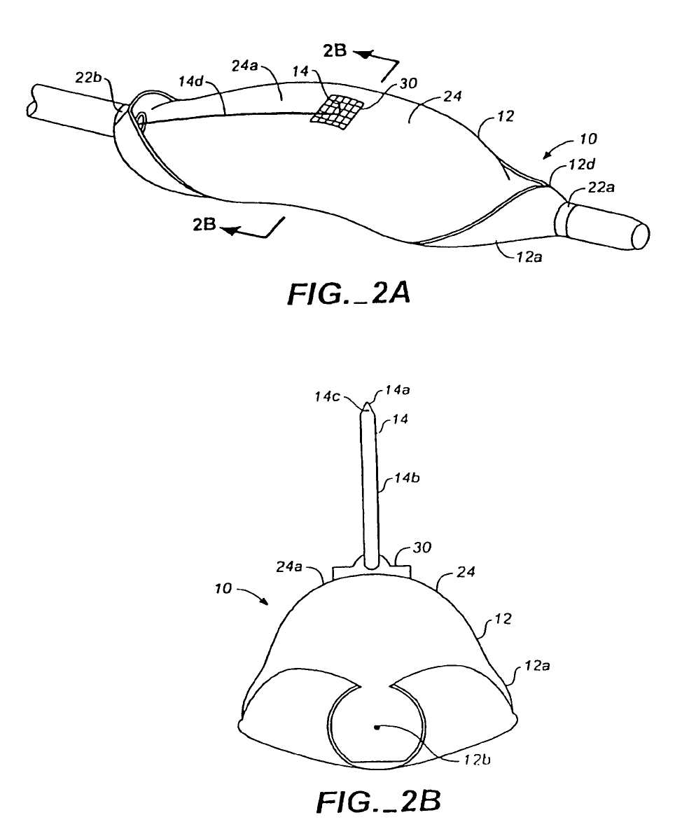

As shown in FIGS. 1A-2B, a microfabricated intravascular catheter 10 includes an actuator 12 having an actuator body 12a and central longitudinal axis 12b. The actuator body more or less forms a C-shaped outline having an opening or slit 12d extending substantially along its length. A microneedle 14 is located within the actuator body, as discussed in more detail below, when the actuator is in its unactuated condition (furled state) (FIG. 1B). The microneedle is moved outside the actuator body when the actuator is operated to be in its actuated condition (unfurled state) (FIG. 2B).

The actuator may be capped at its proximal end 12e and distal end 12f by a lead end 16 and a tip end 18, respectively, of a therapeutic catheter 20. The catheter tip end serves as a means of locating the actuator inside a blood vessel by use of a radio opaque coatings or markers. The catheter tip also forms a seal at the distal end 12f of the actuator. The lead end of the catheter provides the necessary interconnects (fluidic, mechanical, electrical or optical) at the proximal end 12e of the actuator.

Retaining rings 22a and 22b are located at the distal and proximal ends, respectively, of the actuator. The catheter tip is joined to the retaining ring 22a, while the catheter lead is joined to retaining ring 22b. The retaining rings are made of a thin, on the order of 10 to 100 microns (μm), substantially rigid material, such as Parylene (types C, D or N), or a metal, for example, aluminum, stainless steel, gold, titanium or tungsten. The retaining rings form a rigid substantially “C”- shaped structure at each end of the actuator. The catheter may be joined to the retaining rings by, for example, a butt-weld, an ultra sonic weld, integral polymer encapsulation or an adhesive such as an epoxy.

The actuator body further comprises a central, expandable section 24 located between retaining rings 22a and 22b. The expandable section 24 includes an interior open area 26 for rapid expansion when an activating fluid is supplied to that area. The central section 24 is made of a thin, semi-rigid or rigid, expandable material, such as a polymer, for instance, Parylene (types C, D or N), silicone, polyurethane or polyimide. The central section 24, upon actuation, is expandable somewhat like a balloon-device.

The central section is capable of withstanding pressures of up to about 100 psi upon application of the activating fluid to the open area 26. The material from which the central section is made of is rigid or semi-rigid in that the central section returns substantially to its original configuration and orientation (the unactuated condition) when the activating fluid is removed from the open area 26. Thus, in this sense, the central section is very much unlike a balloon which has no inherently stable structure.

The open area 26 of the actuator is connected to a delivery conduit, tube or fluid pathway 28 that extends from the catheter's lead end to the actuator's proximal end. The activating fluid is supplied to the open area via the delivery tube. The delivery tube may be constructed of Teflon© or other inert plastics. The activating fluid may be a saline solution or a radio-opaque dye.

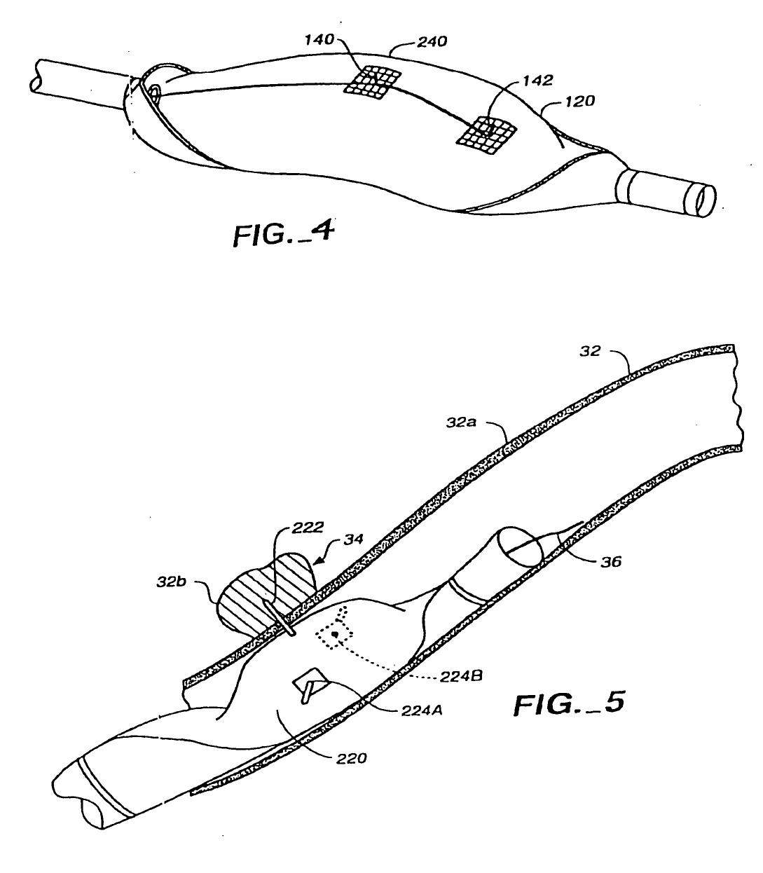

The microneedle 14 may be located approximately in the middle of the central section 24. However, as discussed below, this is not necessary, especially when multiple microneedles are used. The microneedle is affixed to an exterior surface 24a of the central section. The microneedle is affixed to the surface 24a by an adhesive, such as cyanoacrylate. Alternatively, the microneedle maybe joined to the surface 24a by a metallic or polymer mesh-like structure 30 (See FIG. 4F), which is itself affixed to the surface 24a by an adhesive. The mesh-like structure may be-made of, for instance, steel or nylon.

The microneedle includes a sharp tip 14a and a shaft 14b. The microneedle tip can provide an insertion edge or point. The shaft 14b can be hollow and the tip can have an outlet port 14c, permitting the injection of a pharmaceutical or drug into a patient. The microneedle, however, does not need to be hollow, as it may be configured like a neural probe to accomplish other tasks.

As shown, the microneedle extends approximately perpendicularly from surface 24a. Thus, as described, the microneedle will move substantially perpendicularly to an axis of a vessel or artery into which has been inserted, to allow direct puncture or breach of vascular walls.

The microneedle further includes a pharmaceutical or drug supply conduit, tube or fluid pathway 14d which places the microneedle in fluid communication with the appropriate fluid interconnect at the catheter lead end. This supply tube may be formed integrally with the shaft 14b, or it may be formed as a separate piece that is later joined to the shaft by, for example, an adhesive such as an epoxy.

The needle 14 may be a 30-gauge, or smaller, steel needle. Alternatively, the microneedle may be microfabricated from polymers, other metals, metal alloys or semiconductor materials. The needle, for example, may be made of Parylene, silicon or glass. Microneedles and methods of fabrication are described in U.S. application Ser. No. 09/877,653, filed Jun. 8, 2001, entitled “Microfabricated Surgical Device”, assigned to the assignee of the subject application, the entire disclosure of which is incorporated herein by reference.

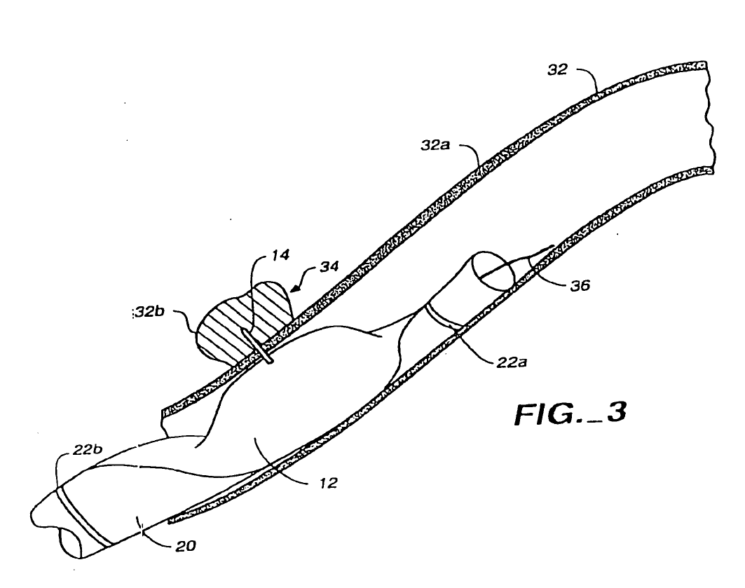

The catheter 20, in use, is inserted through an artery or vein and moved within a patient's vasculature, for instance, a vein 32, until a specific, targeted region 34 is reaches (see FIG. 3). The targeted region 34 may be the site of tissue damage or more usually will be adjacent the sites typically being within 100 mm or less to allow migration of the therapeutic agents. As is well known in catheter-based interventional procedures, the catheter 20 may follow a guide wire 36 that has previously been inserted into the patient. Optionally, the catheter 20 may also follow the path of a previously-inserted guide catheter (not shown) that encompasses the guide wire.

During maneuvering of the catheter 20, well-known methods of fluoroscopy or magnetic resonance imaging (MRI) can be used to image the catheter and assist in positioning the actuator 12 and the microneedle 14 at the target region. As the catheter is guided inside the patient's body, the microneedle remains unfurled or held inside the actuator body so that no trauma is caused to the vascular walls.

After being positioned at the target region 34, movement of the catheter is terminated and the activating fluid is supplied to the open area 26 of the actuator, causing the expandable section 24 to rapidly unfurl, moving the microneedle 14 in a substantially perpendicular direction, relative to the longitudinal central axis 12b of the actuator body 12a, to puncture a vascular wall 32a. It may take only between approximately 100 milliseconds and two seconds for the microneedle to move from its furled state to its unfurled state.

The ends of the actuator at the retaining rings 22a and 22b remain rigidly fixed to the catheter 20. Thus, they do not deform during actuation. Since the actuator begins as a furled structure, its so-called pregnant shape exists as an unstable buckling mode. This instability, upon actuation, produces a large-scale motion of the microneedle approximately perpendicular to the central axis of the actuator body, causing a rapid puncture of the vascular wall without a large momentum transfer. As a result, a microscale opening is produced with very minimal damage to the surrounding tissue. Also, since the momentum transfer is relatively small, only a negligible bias force is required to hold the catheter and actuator in place during actuation and puncture.

The microneedle, in fact, travels so quickly and with such force that it can enter perivascular tissue 32b as well as vascular tissue. Additionally, since the actuator is “parked” or stopped prior to actuation, more precise placement and control over penetration of the vascular wall are obtained.

After actuation of the microneedle and delivery of the cells to the target region via the microneedle, the activating fluid is exhausted from the open area 26 of the actuator, causing the expandable section 24 to return to its original, furled state. This also causes the microneedle to be withdrawn from the vascular wall. The microneedle, being withdrawn, is once again sheathed by the actuator.

Various microfabricated devices can be integrated into the needle, actuator and catheter for metering flows, capturing samples of biological tissue, and measuring pH. The device 10, for instance, could include electrical sensors for measuring the flow through the microneedle as well as the pH of the pharmaceutical being deployed. The device 10 could also include an intravascular ultrasonic sensor (IVUS) for locating vessel walls, and fiber optics, as is well known in the art, for viewing the target region. For such complete systems, high integrity electrical, mechanical and fluid connections are provided to transfer power, energy, and pharmaceuticals or biological agents with reliability.

By way of example, the microneedle may have an overall length of between about 200 and 3,000 microns (μm). The interior cross-sectional dimension of the shaft 14b and supply tube 14d may be on the order of 20 to 250 um, while the tube's and shaft's exterior cross-sectional dimension may be between about 100 and 500 μm. The overall length of the actuator body may be between about 5 and 50 millimeters (mm), while the exterior and interior cross-sectional dimensions of the actuator body can be between about 0.4 and 4 mm, and 0.5 and 5 mm, respectively. The gap or slit through which the central section of the actuator unfurls may have a length of about 4-40 mm, and a cross-sectional dimension of about 100-500 μm. The diameter of the delivery tube for the activating fluid may be about 100 μm. The catheter size may be between 1.5 and 15 French (Fr).

Variations of the invention include a multiple-buckling actuator with a single supply tube for the activating fluid. The multiple-buckling actuator includes multiple needles that can be inserted into or through a vessel wall for providing injection at different locations or times.

For instance, as shown in FIG. 4, the actuator 120 includes microneedles 140 and 142 located at different points along a length or longitudinal dimension of the central, expandable section 240. The operating pressure of the activating fluid is selected so that the microneedles move at the same time. Alternatively, the pressure of the activating fluid may be selected so that the microneedle 140 moves before the microneedle 142.

Specifically, the microneedle 140 is located at a portion of the expandable section 240 (lower activation pressure) that, for the same activating fluid pressure, will buckle outwardly before that portion of the expandable section (higher activation pressure) where the microneedle 142 is located. Thus, for example, if the operating pressure of the activating fluid within the open area of the expandable section 240 is two pounds per square inch (psi), the microneedle 140 will move before the microneedle 142. It is only when the operating pressure is increased to four psi, for instance, that the microneedle 142 will move. Thus, this mode of operation provides staged buckling with the microneedle 140 moving at time t₁, and pressure p₁, and the microneedle 142 moving at time t₂ and P₂, with t₁, and p₁, being less than t₂ and P₂, respectively.

This sort of staged buckling can also be provided with different pneumatic or hydraulic connections at different parts of the central section 240 in which each part includes an individual microneedle.

Also, as shown in FIG. 5, an actuator 220 could be constructed such that its needles 222 and 224A move in different directions. As shown, upon actuation, the needles move at angle of approximately 90° to each other to puncture different parts of a vessel wall. A needle 224B (as shown in phantom) could alternatively be arranged to move at angle of about 180° to the needle 224A.

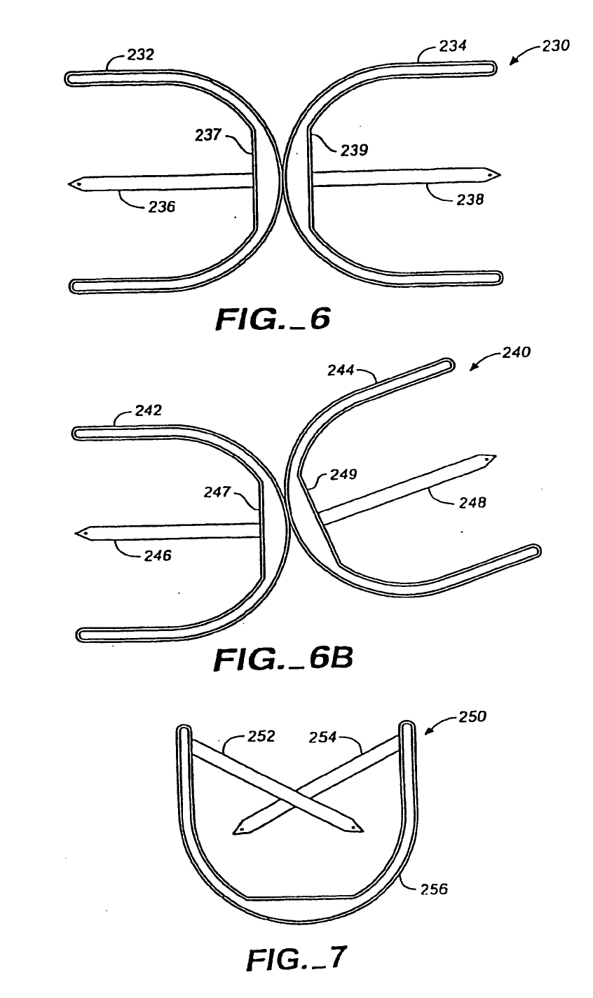

Moreover, as shown in FIG. 6A, in another embodiment, an actuator 230 comprises actuator bodies 232 and 234 including needles 236 and 238, respectively, that move approximately horizontally at angle of about 180° to each other. Also, as shown in FIG. 7B, an actuator 240 comprises actuator bodies 242 and 244 including needles 242 and 244, respectively, that are configured to move at some angle relative to each other than 90° or 180°. The central expandable section of the actuator 230 is provided by central expandable sections 237 and 239 of the actuator bodies 232 and 234, respectively. Similarly, the central expandable section of the actuator 240 is provided by central expandable sections 247 and 249 of the actuator bodies 242 and 244, respectively.

Additionally, as shown in FIG. 7, an actuator 250 may be constructed that includes multiple needles 252 and 254 that move in different directions when the actuator is caused to change from the unactuated to the actuated condition. The needles 252 and 254, upon activation, do not move in a substantially perpendicular direction relative to the longitudinal axis of the actuator body 256.

The above catheter designs and variations thereon, are described in published U.S. Patent Application Nos. 2003/005546 and 2003/0055400, the full disclosures of which are incorporated herein by reference. Co-pending application Ser. No. 10/350,314, assigned to the assignee of the present application, describes the ability of substances delivered by direct injection into the adventitial and pericardial tissues of the heart to rapidly and evenly distribute within the heart tissues, even to locations remote from the site of injection. The full disclosure of that co-pending application is also incorporated herein by reference. An alternative needle catheter design suitable for delivering the drug of the present invention will be described below. That particular catheter design is described and claimed in co-pending application Ser. No. 10/393,700 (Attorney Docket No. 021621-001500 U.S.), filed on Mar. 19, 2003, the full disclosure of which is incorporated herein by reference.

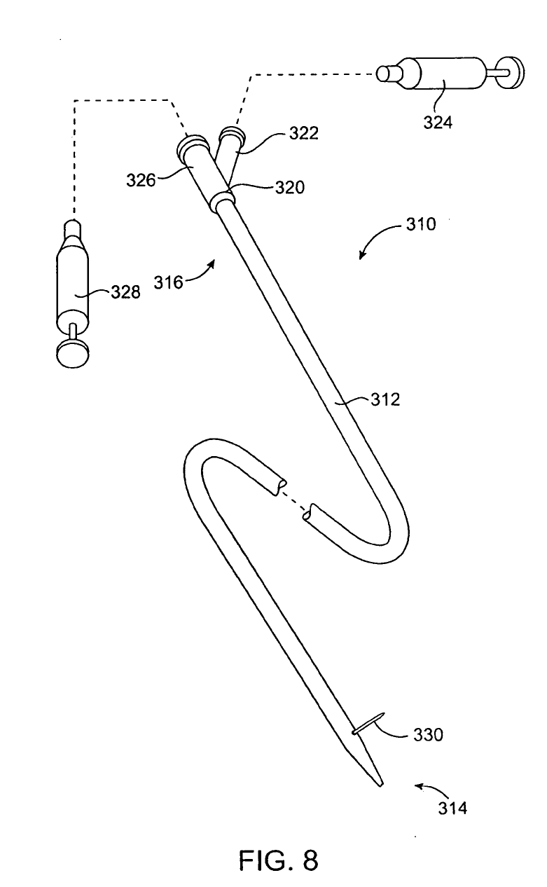

Referring now to FIG. 8, a needle injection catheter 310 constructed in accordance with the principles of the present invention comprises a catheter body 312 having a distal end 314 and a proximal 316. Usually, a guide wire lumen 313 will be provided in a distal nose 352 of the catheter, although over-the-wire and embodiments which do not require guide wire placement will also be within the scope of the present invention. A two-port hub 320 is attached to the proximal end 316 of the catheter body 312 and includes a first port 322 for delivery of a hydraulic fluid, e.g., using a syringe 324, and a second port 326 for delivering the pharmaceutical agent, e.g., using a syringe 328. A reciprocatable, deflectable needle 330 is mounted near the distal end of the catheter body 312 and is shown in its laterally advanced configuration in FIG. 8.

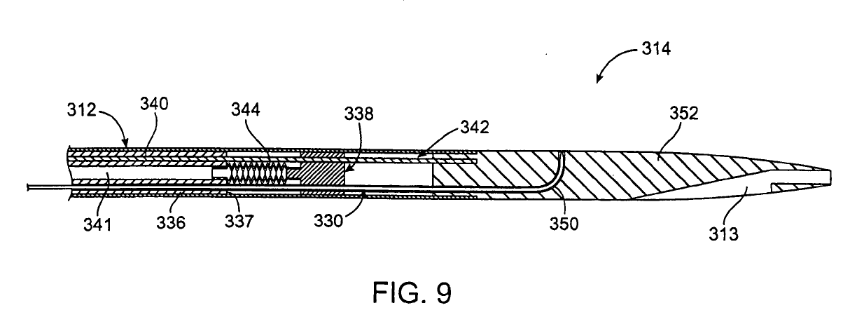

Referring now to FIG. 9, the proximal end 314 of the catheter body 312 has a main lumen 336 which holds the needle 330, a reciprocatable piston 338, and a hydraulic fluid delivery tube 340. The piston 338 is mounted to slide over a rail 342 and is fixedly attached to the needle 330. Thus, by delivering a pressurized hydraulic fluid through a lumen 341 tube 340 into a bellows structure 344, the piston 338 may be advanced axially toward the distal tip in order to cause the needle to pass through a deflection path 350 formed in a catheter nose 352.

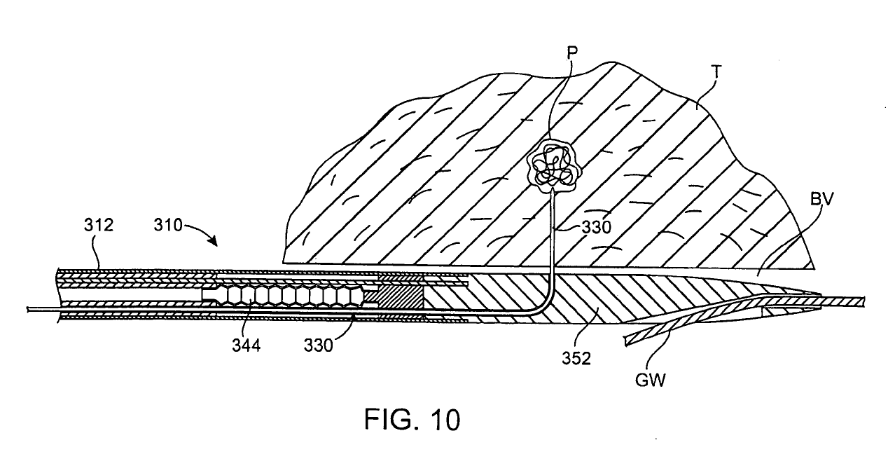

As can be seen in FIG. 10, the catheter 310 may be positioned in a coronary blood vessel BV, over a guide wire GW in a conventional manner. Distal advancement of the piston 338 causes the needle 330 to advance into luminal tissue T adjacent to the catheter when it is present in the blood vessel. The drug may then be introduced through the port 326 using syringe 328 in order to introduce a plume P of drug in the cardiac tissue, as illustrated in FIG. 10. The plume P will be within or adjacent to the region of tissue damage as described above.

The needle 330 may extend the entire length of the catheter body 312 or, more usually, will extend only partially in drug delivery lumen 337 in the tube 340. A proximal end of the needle can form a sliding seal with the lumen 337 to permit pressurized delivery of the drug through the needle.

The needle 330 will be composed of an elastic material, typically an elastic or super elastic metal, typically being nitinol or other super elastic metal. Alternatively, the needle 330 could be formed from a non-elastically deformable or malleable metal which is shaped as it passes through a deflection path. The use of non-elastically deformable metals, however, is less preferred since such metals will generally not retain their straightened configuration after they pass through the deflection path.

The bellows structure 344 may be made by depositing by parylene or another conformal polymer layer onto a mandrel and then dissolving the mandrel from within the polymer shell structure. Alternatively, the bellows 344 could be made from an elastomeric material to form a balloon structure. In a still further alternative, a spring structure can be utilized in, on, or over the bellows in order to drive the bellows to a closed position in the absence of pressurized hydraulic fluid therein.

After the drug is delivered through the needle 330, as shown in FIG. 10, the needle is retracted and the catheter either repositioned for further agent delivery or withdrawn. In some embodiments, the needle will be retracted simply by aspirating the hydraulic fluid from the bellows 344. In other embodiments, needle retraction may be assisted by a return spring, e.g., locked between a distal face of the piston 338 and a proximal wall of the distal tip 352 (not shown) and/or by a pull wire attached to the piston and running through lumen 341.

While the above is a complete description of the preferred embodiments of the invention, various alternatives, modifications, and equivalents may be used. Therefore, the above description should not be taken as limiting the scope of the invention which is defined by the appended claims.

1. A method for treating a patient suffering from a cardiac arrhythmia, said method comprising delivering a class III anti-arrhythmic drug to peri-adventitial tissue of the patient's heart.

2. A method as in claim 1, wherein delivering comprises injecting the anti-arrhythmic drug through the endothelium of a blood vessel.

3. A method as in claim 1, wherein delivering comprises injecting the anti-arrhythmic drug transmyocardially.

4. A method as in any one of claims 1 to 3, wherein the anti-arrhythmic drug is amiodarone.

5. A method as in any one of claims 2 and 4, wherein the blood vessel is an artery.

6. A method as in any one of claims 2 and 4, wherein the blood vessel is a vein.

7. A method as in any one of claims 2 and 4 to 6, wherein injecting comprises advancing a needle from a lumen of the blood vessel to the location beyond the endothelium and infusing the drug through the needle.

8. A method as in claim 7, wherein the needle is advanced into a perivascular space beyond the outside of the endothelium.

9. A method as in claim 8, wherein the needle is advanced into the adventitia surrounding the blood vessel.

10. A method as in any one of claims 2 to 9, wherein the class III anti-arrhythmic drug is injected in an amount sufficient to permeate circumferentially around the endothelium and into the adventitia over an axial length of at least 1 cm.

11. A method as in any one of claims 2 and 4 to 10, wherein the needle is advanced in a radial direction to a depth in the adventitia equal to at least 10% of the mean luminal diameter at the blood vessel location.

12. A method as in claim 11, wherein the depth is a distance in the range from 10% to 150% of the mean luminal diameter.

13. A method as in any one of claims 1 to 12, wherein the tissue is cardiac which has been damaged by a myocardial infarction.

14. A method for treating a patient suffering from a cardiac arrhythmia, said method comprising:
advancing a needle from a lumen of the blood vessel to the location beyond the endothelium of the blood vessel; and
injecting amiodarone through the needle into tissue at a location beyond the endothelium of the vessel.

15. A method as in claim 14, wherein the blood vessel is a coronary artery.

16. A method as in claim 14, wherein the blood vessel is a coronary vein.

17. A method as in claim 14, wherein the needle is advanced into a perivascular space beyond the outside of the endothelium.

18. A method as in claim 17, wherein the needle is advanced into the adventitia and/or periadventitial surrounding the blood vessel.

19. A method as in any one of claims 14-18, wherein the amiodarone is injected in an amount sufficient to permeate circumferentially around the endothelium and into the adventitia over an axial length of at least 1 cm.

20. A method as in any one of claims 14 to 19, wherein the needle is advanced in a radial direction to a depth in the adventitia equal to at least 10% of the mean luminal diameter at the blood vessel location.

21. A method as in claim 20, wherein the depth is a distance in the range from 10% to 150% of the mean luminal diameter.

22. A method as in any one of claims 14-12, wherein the cardiac tissue has been damaged on a myocardial infarction.

23. A system for treating cardiac arrhythmias, said system comprising:
an amount of a class III anti-arrhythmic drug selected to inhibit a cardiac arrhythmia when delivered to a location beyond the endothelium of a blood vessel; and
an intravascular catheter having a needle for injecting the class III anti-arrhythmic drug into a location beyond the endothelium of a blood vessel.

24. A system as in claim 23, wherein the class III anti-arrhythmic drug comprises amiodarone.
